(19) United States
(12) Patent Application Publication
(10) Pub. No.: US 2014/0005723 A1
(43) Pub. Date: Jan. 2, 2014

(54) INTRA SPINOUS PROCESS AND METHOD OF BONE GRAFT PLACEMENT

(71) Applicants: Jawad A. Shah, Flushing, MI (US); Richard H. Smith, Dexter, MI (US); Amer Iqbal, Grand Blanc, MI (US)

(72) Inventors: Jawad A. Shah, Flushing, MI (US); Richard H. Smith, Dexter, MI (US); Amer Iqbal, Grand Blanc, MI (US)

(73) Assignee: IINN, Inc., Flint, MI (US)

(21) Appl. No.: 14/004,626

(22) PCT Filed: Mar. 12, 2012
(86) PCT No.: PCT/US12/28727

§ 371 Date: Sep. 11, 2013

(30) Foreign Application Priority Data
Mar. 11, 2011 (US) 61451835

(51) Int. Cl. A61B 17/70; A61B 17/88

(52) U.S. Cl. CPC A61B 17/7062; A61B 17/88
USPC 606/248; 606/279

(57) ABSTRACT

A spinal fixation plate for posterior fixation, including a plate body having flattened flanges extending along sides of the plate body, the flanges being interconnected by a convex arcuate process. A method of fixing vertebrae, including the steps of disposing a spinal fixation plate upon dorsal surfaces of adjoining vertebrae, wherein flanges of the spinal fixation plate extend parallel to the spinal column and rest upon superior articular processes of the adjoining vertebrae, and anchoring the spinal fixation plate to the vertebrae with at least one fastener by disposing the fastener through at least one aperture of the spinal fixation plate into the vertebrae.

### TECHNICAL FIELD

The present invention relates to spinal fixation plates. More specifically, the present invention relates to a spinal fixation plate that includes an arcuate portion to circumvent the spinous processes and allow the plate to be anchored on the dorsum of the spine, and to the method of using this plate in spinal fusion involving bone grafts.

### BACKGROUND OF THE INVENTION

Spinal fixation has become a common approach in treating spinal disorders, fractures, and for fusion of vertebrae. Surgery of this kind is indicated when the patient experiences chronic pain due to abnormal motion of the vertebrae, where immobilizing the vertebrae can provide pain relief. The aim of the surgery is to stabilize adjacent vertebrae that are the foci for a variety of medical conditions. Spinal fixation can be used alone or in conjunction with spinal fusion in the treatment of degenerative disc disease and other conditions, including spinal disc herniation, discogenic pain, spinal tumor, vertebral fracture, scoliosis, kyphosis, spondylolisthesis, spondylosis, and other degenerative spinal conditions or any condition that causes instability of the spine.

Bone fixation plates are commonly used for spinal fixation. Typical bone fixation plates have a relatively flat, rectangular body portion including a plurality of apertures extending therethrough. Fasteners such as screws are inserted through the apertures and firmly tightened to secure the bone fixation plate to the bone or bones to be fixed. There are numerous examples of bone fixation plates existing in the art. These are illustrated in U.S. Pat. No. 5,364,399 to Lowery et al., U.S. Pat. No. 5,601,553 to Trebing et al., U.S. Pat. No. 6,017,345 to Richelsoph, U.S. Pat. No. 6,152,927 to Farris et al., U.S. Pat. No. 6,235,034 B1 to Bray, U.S. Pat. No. 6,139,550 to Michelson, and U.S. Pat. No. 6,258,089 B1 to Campbell et al. The above referenced patents are cited as examples illustrating the general state of the art with regard to bone fixation plate technology. Generally, these types of devices can be utilized for the fixation of any bone or bones, but are more particularly suited for the fixation of the spine with regard to the cervical, lumbar and/or thoracic regions.

One method of spinal fixation is to approach the spine from the anterior or anterior-lateral side and to use screws to solidly mount the fixation plate to the affected vertebrae. This anterior stabilization approach is commonly used in cases of trauma, tumors, and degenerative conditions. As previously discussed, a fixation plate is often used as an adjunct to spinal fusion, wherein graft material is placed between or along adjacent vertebrae to permanently fuse the vertebrae together. The graft material can consist of bone grafts obtained from other bones in the patient's body or from cadaver bones, along with bone growth factors and bulking agents. Fixation plates and other fixation hardware keep the affected vertebrae immobilized and aligned while the grafts grow and fusion develops.

In many conditions, an anterior or anterior-lateral installation is either not optimal or not practical, and a posterior approach or a combined posterior-anterior approach is indicated. It has not been possible, however, to perform posterior stabilization with the type of spinal fixation plate that has proven effective in anterior stabilization; that is, a solid plate which anchors to the pedicles of two or more vertebrae by means of bone screws or other fasteners. The generally flattened spinal fixation plates of the prior art cannot be anchored upon the dorsal aspect of the vertebrae because they are blocked by the presence of the spinous processes that project posteriorly from the vertebrae.

A well known alternative strategy for posterior stabilization is to place a clamp laterally about the spinous processes of adjacent vertebrae. The clamp is tightened into place by a bolt or rod traversing the space between adjacent spinous processes. This approach is embodied in spinous process clamps such as the HORIZON SPIRE™ spinous process plate (Medtronic, Minneapolis Minn.) and the AFFIX II™ spinous process plate (NuVasive(R), San Diego Calif.).

Such spinous process clamps have important drawbacks. Their use depends on the presence of intact spinous processes. They cannot be employed when spinous processes are damaged by disease or trauma or have been removed during the course of therapeutic laminotomy or laminectomy. Spinous process clamps can also be difficult to secure when there are large anatomical differences between neighboring spinous processes. The clamps also lack the physical robustness of a solid plate that mechanically couples two adjoining vertebrae by means of a multiplicity of screws, an arrangement that provides efficient load sharing and transfer and attendant stability. Furthermore, spinous process clamps join and immobilize adjacent spinous processes and are therefore liable to fracture the spinous processes by exerting torque on the processes during normal body movements and, more dramatically, during a fall.

Spinal process clamps of the prior art can allow ingrowth of bone into the spinal canal, as they include no features that maintain a bone graft in the optimal position for successful fusion. They provide no covering for the thecal sac, and therefore do not limit overgrowth of tissue. In addition, the spinal fixation devices of the prior art do not provide structures that promote fusion by encouraging the adhesion and growth of grafted bone.

It frequently is the case that spinal fusion surgeries must be repeated on patients. This can be due to the degenerative nature of the disease, repeated injury, or other reasons. As an example, a study published in Spine (Sep. 1, 2007), analyzing about 2500 patients undergoing lumbar surgery during 1990-93 and 1997-2000, found an increase in repeated spinal fusion surgeries over time. Because conditions such as degenerative disc disease or spinal tumors are progressive, and there is a frequent occurrence of injury or other conditions relating to additional medical situations that require spinal fusion, repeated surgical intervention can be required.

Accordingly, there is a need for a spinal fixation plate that can be attached to the dorsum of the spine from a posterior approach, and that can be fixed in place with fasteners entering the pedicles from the generally dorsal aspect of the vertebrae. There is a need for a spinal fixation plate that can be employed for posterior spinal fixation despite damage to or absence of the spinal processes, or when the spinal processes show large anatomical variation. There is also a need for a spinal fixation plate that provides surfaces to promote bone implant growth and inter-vertebral fusion and provides covering for the thecal sac. Finally, there is a need for a posterior spinal fixation plate with features that facilitate repeated surgeries by allowing simpler and more rapid insertion and removal, and reduced damage to muscle and fascia, than the devices of the prior art.

Screws often are used to immobilize part of the spine to assist fusion by holding bony structures together. Fixation by means of screws is most often accomplished using access holes drilled through the pedicles. By using such placement, the entire shaft of each screw is affixed in bone, thereby minimizing discomfort or damage to the spinal cord or intravertebral discs. Pedicle screws typically are polyaxial, thereby allowing placement at a variety of angles as indicated in each case. The spinal fixation plate described herein can be stabilized with appropriate pedicle screws as are available.

### SUMMARY OF THE INVENTION

The present invention provides for a spinal fixation plate for posterior fixation, including a plate body having flattened flanges extending along sides of the plate body, the flanges being interconnected by a convex arcuate process.

The present invention also provides for a method of fixing vertebrae, including the steps of disposing a spinal fixation plate upon dorsal surfaces of adjoining vertebrae, wherein flanges of the spinal fixation plate extend parallel to the spinal column and rest upon superior articular processes of the adjoining vertebrae, and anchoring the spinal fixation plate to the vertebrae with at least one fastener by disposing the fastener through at least one aperture of the spinal fixation plate into the vertebrae.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a top view of a spinal fixation plate in accordance with the present invention;

FIG. 2A is a perspective view of a spinal fixation plate in accordance with the present invention;

FIG. 2B. is a perspective view of an alternative embodiment of the spinal fixation plate.

FIG. 3 is a longitudinal cross section of an embodiment of a spinal fixation plate in accordance with the present invention;

FIG. 4A is a perspective view of a grooved embodiment of a spinal fixation plate in accordance with the present invention;

FIG. 4B is a perspective view of an alternative grooved embodiment of the spinal fixation plate.

FIG. 5A is a superior cross section of a vertebra showing the attachment thereto of a spinal fixation plate in accordance with the present invention;

FIG. 5B is a superior cross section of a vertebra showing an alternative attachment thereto of a spinal fixation plate;

FIG. 5C is a superior cross section of a vertebra showing a spinal fixation plate attached to vertebral laminae;

FIG. 6A is a diagrammatic lateral view of a portion of a spinal column showing the attachment thereto of an embodiment of the spinal fixation plate of the present invention (represented by dashed lines);

FIG. 6B is a diagrammatic lateral view of a portion of a spinal column showing an alternative attachment thereto of an embodiment of the spinal fixation plate of the present invention (represented by dashed lines);

FIG. 7 is a side view of an alternative embodiment of the present invention featuring curvature transverse to the longitudinal axis;

FIG. 8A is a top elevation of a compressible spacer;

FIG. 8B is a cross section of a compressible spacer;

FIG. 8C is a diagrammatic longitudinal section of a vertebra showing an embodiment of the present invention that includes compressible spacers;

FIG. 9 is a posterior elevation of a section of spine showing the use of a spinal fixation plate in conjunction with bone graft, rods, and polyaxial pedicle screws;

FIG. 10 is a longitudinal cross section of an alternative embodiment of the present invention showing a gripping subassembly in slack condition;

FIG. 11 is a longitudinal cross section showing detail of a bar receiver of a gripping subassembly in accord with the present invention, with the gripping subassembly in tightened condition;

FIG. 12A is a superior view of a vertebra showing the attachment thereto of a spinal fixation plate including a gripping subassembly spanning the intervertebral space, the gripping subassembly being in slack condition;

FIG. 12B is a superior view of a vertebra showing the attachment thereto of a spinal fixation plate including a gripping subassembly spanning the intervertebral space, the gripping subassembly being in tightened condition;

FIG. 13 is a posterior cutaway view of a spinal fixation plate including a gripping subassembly in tightened condition about the spinal processes of two adjacent vertebrae; and

FIG. 14 is a diagrammatic lateral view of a portion of a spinal column showing the attachment thereto of an embodiment of the spinal fixation plate including spiked projections and a gripping subassembly, with the arcuate process of the plate represented as transparent.

### DETAILED DESCRIPTION OF THE INVENTION

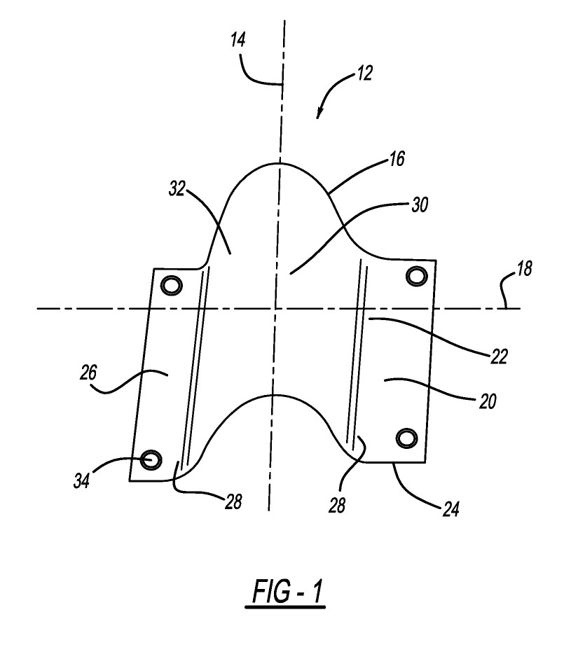

A spinal fixation plate constructed in accordance with the present invention is generally indicated by 12 in the figures. As shown in FIG. 1, generally the fixation plate 12 is a substantially elongated plate body 12 with a longitudinal axis 14 defined by two opposed ends 16, and a lateral axis 18 defined by two opposed sides 20. The fixation plate 12 includes an upper surface 22, and an opposed lower surface 24. The fixation plate 12 also includes flattened flanges 26, each flange extending along each of the sides 20. The flanges 26 are interconnected by an arcuate process 28.

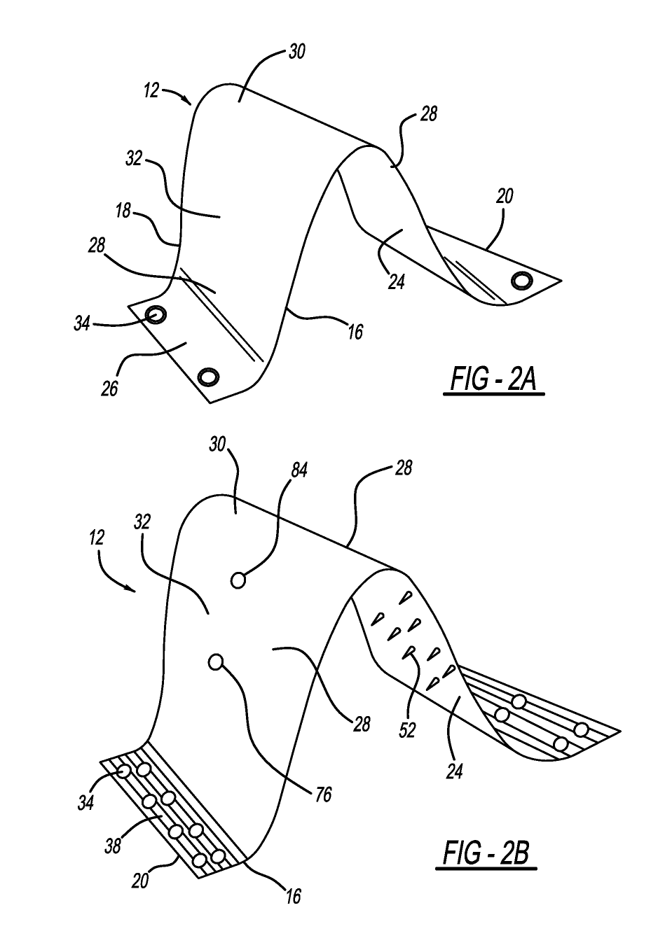

The arcuate process 28 is convex in cross section and describes an arc with its apex 30 generally at the midpoint of the lateral axis 18, indicated in FIG. 2. The arcuate process extends along the length of the longitudinal axis 14 of the fixation plate 12. The shoulders 32 of the arcuate process 28 slope outward to join seamlessly with the flanges 26. The flanges 26 are generally flat and oriented generally parallel to the lateral axis 18 of the fixation plate 12.

It will be understood by those skilled in the art that the following properties of the fixation plate can vary according to the number, size, shape, and curvature of the vertebrae to be spanned by the fixation plate 12: The angle included by the apex 30 of the arcuate process 28, the angle at which the arcuate process 28 meets the flanges 26, the angle of the flanges 26 relative to the lateral axis 18, and the relative proportions of the flanges 26 to the arcuate process 28. The arcuate process 28 is preferably but not necessarily symmetrical about the longitudinal axis 14 of fixation plate 12.

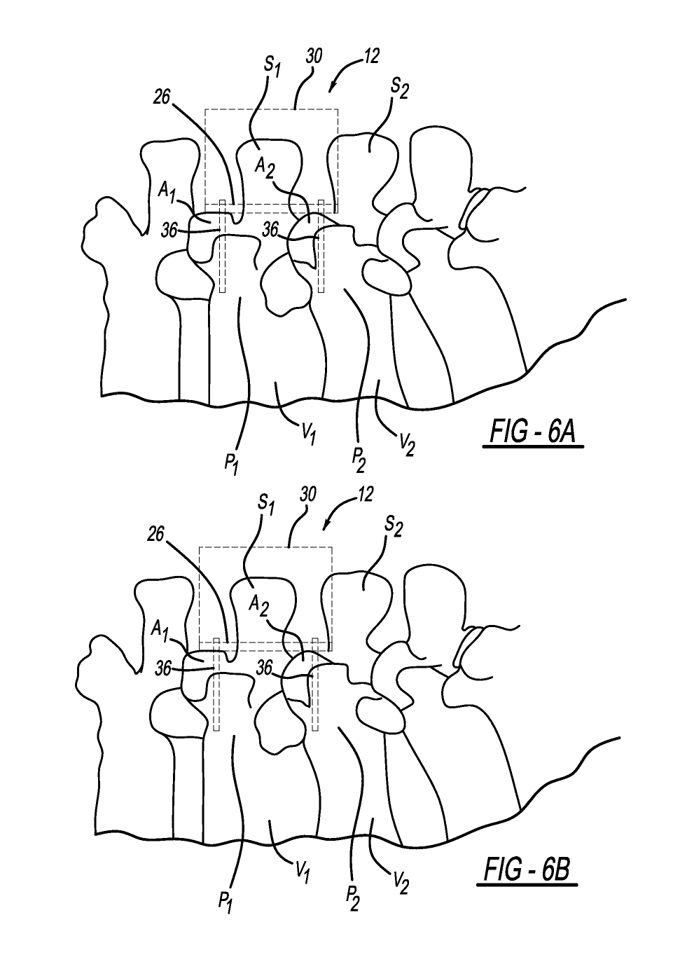

The fixation plate 12 includes at least one aperture 34 extending through the upper surface 22 and the lower surface 24 of the fixation plate 12. The upper surface 22 is the location of the initial insertion of fastening means, preferably pedicle screws 36, and is not touching any bone surface thereon. The lower surface 24 is closest to the spinal column and typically touches the surface of the bones to which the fixation plate 12 is affixed. Preferably there are four apertures, for the insertion of two bone screws 36 into each of two adjoining vertebrae as shown in FIG. 6. The number of apertures 34 can however vary according to design, location and severity of fixation desired. For example, apertures can be situated in two ranks, with more than one aperture per rank, as illustrated in FIGS. 1B, 2B, and 3B. It will be understood that other patterns of distribution of apertures 34 are within the scope of the present invention, and that not all apertures 34 will necessarily be employed in the insertion of pedicle screws 36 or other fastening means. Apertures 34 are preferably disposed in the flanges 26 proximate to each end 16, but they can additionally or alternatively be disposed in the shoulders 32 of the arcuate process 28 if warranted by suitable opportunities for anchorage.

The terms “aperture” or “apertures” 34 as used herein, are meant to include, but are not limited to, any circular hole, oblong hole, slot, elongated slot, through hole, void, and any other similar opening. The aperture 34 should be large enough to accommodate at least a shaft of a pedicle screw 36 and preferably the entire screw head. The aperture 34 is not necessarily limited to just the size of the pedicle screw 36 and screw head. The aperture 34 can be larger than the screw head, but also have a spherical seat or other similar, machined portion on the fixation plate 12 to prevent the screw from passing completely through the aperture 34. Additionally, the aperture 34 can be an elongated slot wherein the screw 36 is capable of sliding within the slot.

The fixation plate 12 of the present invention is preferably constructed of titanium but stainless steel and other metal alloys can be used, as long as they have sufficient strength and corrosion resistance, and are non-reactive and non-antigenic to biological systems. Optionally, fixation plate 12 can be provided with an elastomeric coating (not shown), designed so to minimize host response and tissue adhesion, thereby reducing trauma to the patient in the event of re-operation. Such coating utilizes an FDA-approved substance, examples of which include silicone elastomers, which can also incorporate fillers of fumed silica, or other biocompatible formulations. Substances of this kind are commonly used as protective coatings in other implanted medical devices, such as pacemakers and heart valves. Said elastomeric coating can be applied to fixation plate 12 by a variety of means, including dip-coating, spraying, and lamination, as appropriate to the properties of the coating substance. The coating can be applied to any surface of fixation plate 12, preferably to surfaces that, due to practice in the art, are demonstrated to support unwanted tissue adhesion and growth in the absence of said coating, such that the use of the coating is shown to reduce said tissue adhesion and growth.

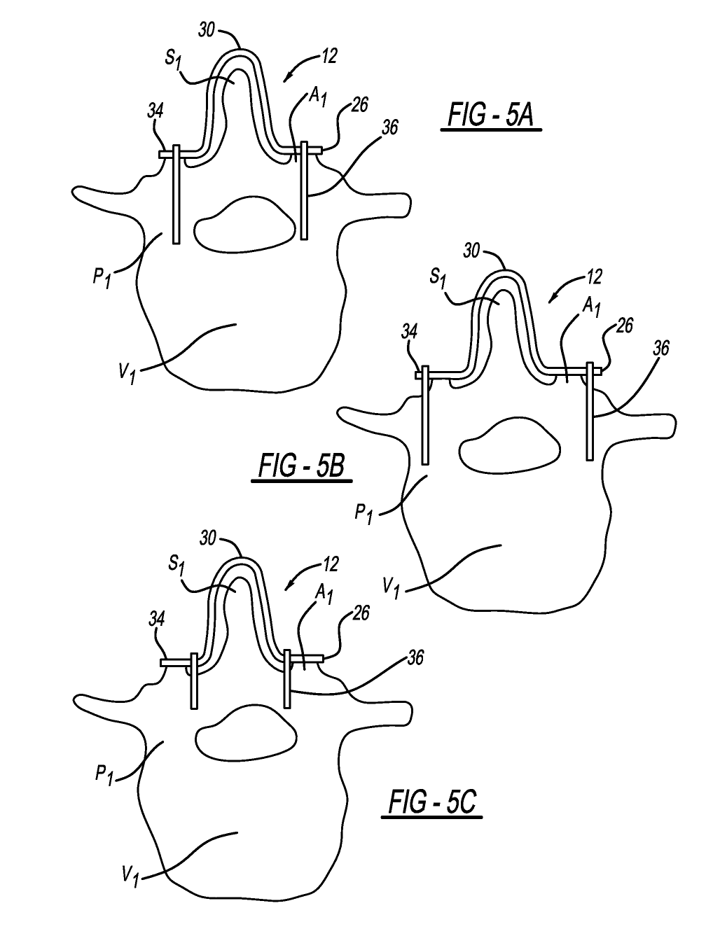

In use, the fixation plate 12 is preferably deployed as shown in FIGS. 5 and 6. The fixation plate 12 is disposed upon the dorsal surfaces of adjoining vertebrae V1 and V2 to be fixed, with the flanges 26 extending parallel to the spinal column and the lower surface 24 facing the vertebrae V1 and V2. The flanges 26 rest upon the superior articular processes A1 and A2 of the adjoining vertebrae V1 and V2. The apex 30 and shoulders 32 of the arcuate process 30 of the fixation plate 12 do not contact the spinous processes S1 and S2 of the vertebrae V1 and V2.

The fixation plate 12 is anchored to the vertebrae by means of at least one fastener, preferably a pedicle screw 36, disposed through at least one aperture 34. The apertures 34 can be situated directly upon the superior articular processes A1 and A2 so that the pedicle screws 36 pass through the dorsal aspect of the superior articular processes A1 and A2 and into the pedicles P1 and P2, as shown in FIGS. 5A and 6A. Alternatively, the apertures 34 can be situated lateral to the superior articular processes A1 and A2 so that the pedicle screws 36 bypass the superior articular processes A1 and A2, and pass directly into each pedicle P1 and P2 from the dorsal aspect of each vertebra V1 and V2, as shown in FIGS. 5A and 6B. Pedicle screws can also be disposed in the laminae, L1 and L2, as shown in FIG. 5C. Preferably, the pedicle screws 36 are disposed within previously drilled holes in the pedicles P1 and P2.

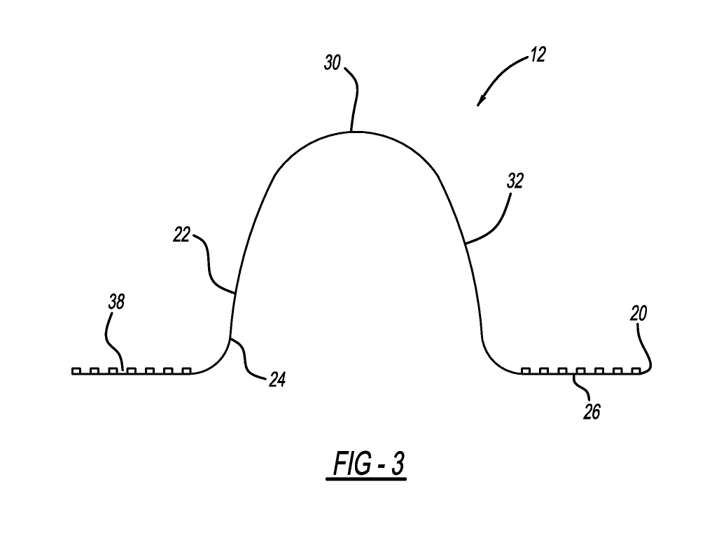

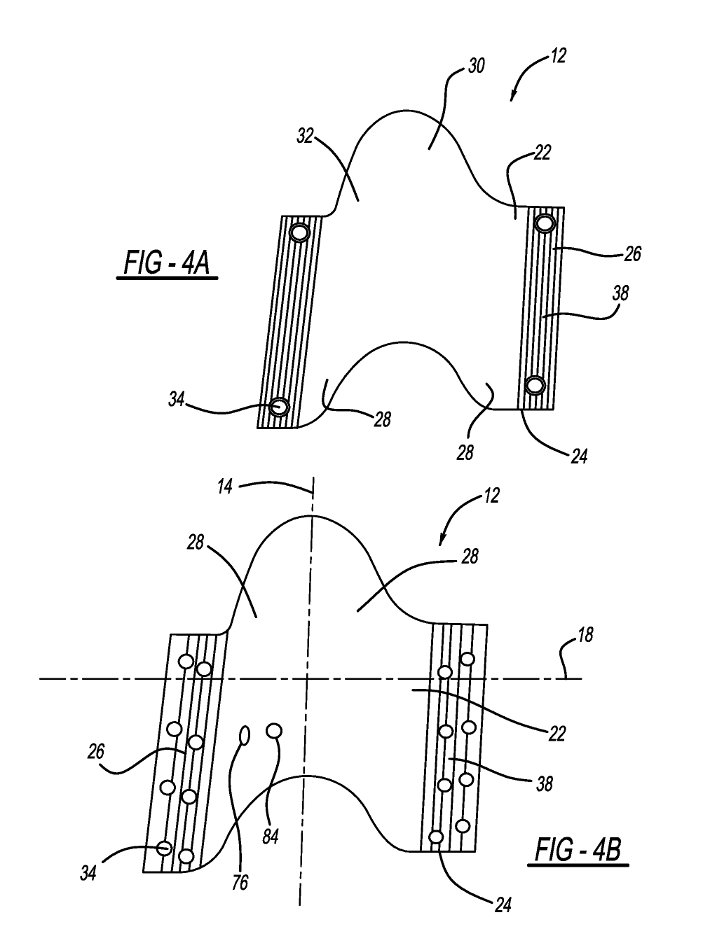

In a preferred embodiment of the invention, the fixation plate 12 includes a multiplicity of grooves 38 disposed substantially on the upper surface 24, as in FIG. 3, but preferably in the region defined by flanges 26 and the lateral axes 18, and extending along the longitudinal axis 14, as shown in FIG. 3 and FIG. 4. The grooves 38 can be oriented at any angle to the longitudinal axis 14, but preferably are parallel to the longitudinal axis 14. In operation, the grooves 38 can be seeded with appropriate bone graft materials, which can include autografts, allografts, or bone graft substitutes. The grooves 38 provide surfaces for bone fusion to occur.

Additionally or alternatively, bone growth factors can be placed in the grooves 38 to provide local, targeted encouragement of bone growth. The preferred bone growth factor is recombinant human bone morphogenetic protein (rhBMP-2), but it can be any suitable growth factor known in the art. The grooves can also contain graft extenders and bulking agents. Preferably, this includes tricalcium phosphate/hydroxyapatite (TCP-HA), but can include any graft extender or bulking agent known in the art.

Placement of bone graft materials, growth factors, extenders, bulking agents, and combinations thereof within the grooves 38 promotes their concentration, secure placement, and direct contact with the decorticated laminae or other bony structures, of adjacent host vertebrae between the transverse processes. Such contact allows and promotes vascularization to occur during bone repair and graft incorporation.

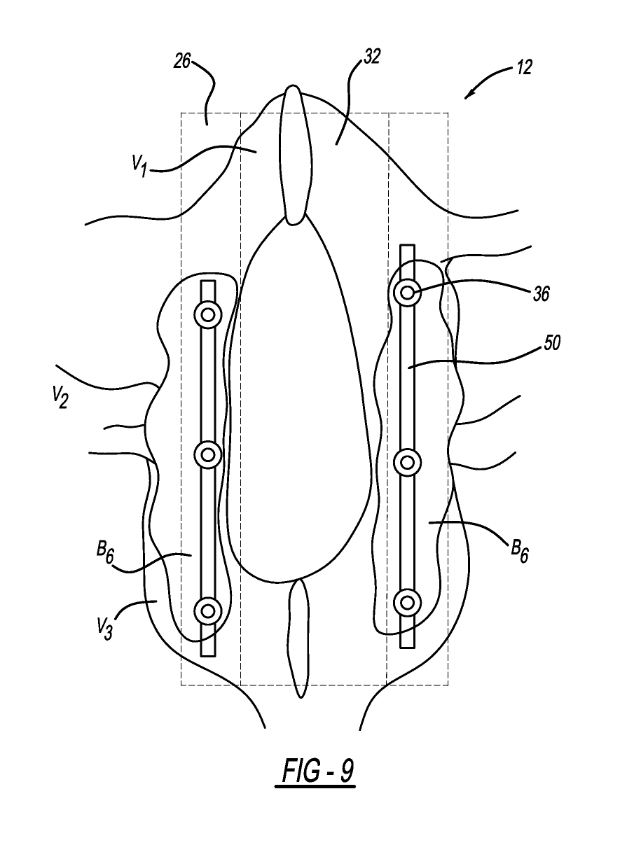

Alternatively, fixation plate 12 is provided with a smooth, ungrooved lower surface, wherein bone graft materials BG can be placed along the flanges 26 of the fixation plate 12, and placed in direct contact with decorticated host bone, as shown in FIG. 9.

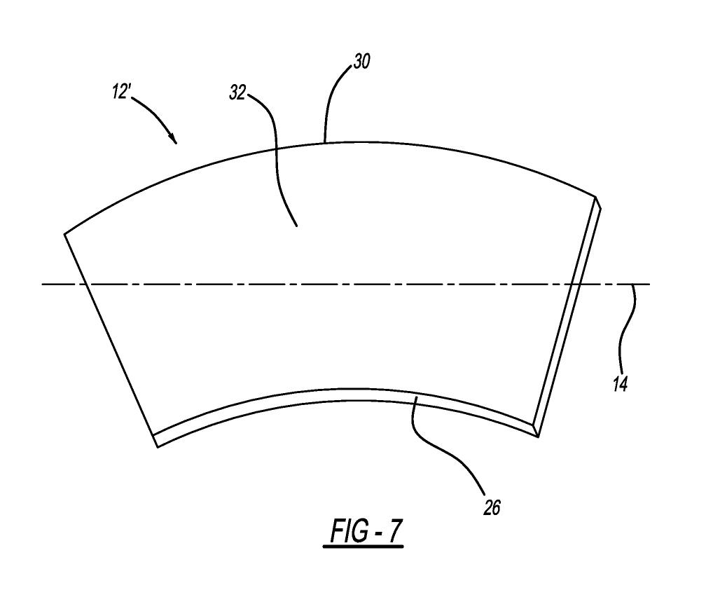

In an alternative further embodiment of the present invention, shown in FIG. 7, the fixation plate 12 is arcuately shaped or curved transverse to the longitudinal axis 14 to conform the fixation plate 12 to the curvature of the vertebrae.

The fixation plate 12 of the present invention is not necessarily of uniform thickness, composition, or rigidity throughout its structure. In one alternative embodiment, the areas surrounding apertures 34 are more flexible than other areas of the structure (not shown). Such differential flexibility can be achieved, for example, by machining the areas around the apertures 34 to be thinner that other areas of the structure, or by fabricating the fixation plate 12 so that a soft alloy is localized about the apertures 34. The flexible areas around the apertures can conform closely to irregular bone surfaces, optimizing the tightness of the anchorage of the fixation plate 12 to the bone.

Conversely, fixation plate 12 can be differentially stiffened in areas where greater resistance to mechanical stress is needed. Differential stiffening of specific areas can be accomplished, for example, by inflecting these areas into ridges (not shown). Such ridges can, for example, be milled into the surface of the fixation plate 12.

The spinal fixation plate 12 is not necessarily solid throughout its structure. Areas of the shoulders 32, flanges 26, and apex 30 can include cut out areas (not shown). Such cut-outs can increase the flexibility and lightness of the spinal fixation plate 12 and reduce the cost of its manufacture. Cut-outs are of course limited to areas wherein they do not degrade the structural strength of the fixation plate 12.

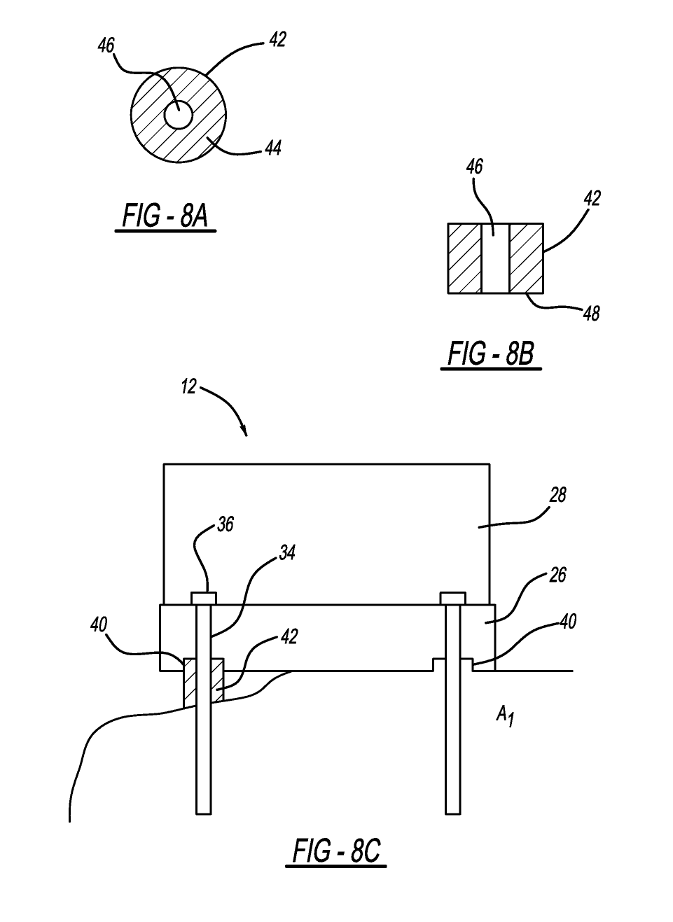

In another embodiment, the lower surface 24 of at least one flange 26 includes at least one recess 40 for the seating of at least one compressible spacer 42. The spacers 42 enhance the anchorage of fixation plate 12 to irregular bone surfaces with which the flanges 26 do not closely conform. The spacers 42 fill gaps between the lower surface 24 of a flange 26 and a bone surface. Preferably the recesses 40 are circular and are situated concentrically about the apertures 34 as shown in FIG. 8C. Each spacer 42 is circular in form, with a diameter equal to or less than that of the recess 40 in which it is seated. Each spacer 42 also defines a central perforation 44. The spacer 42 is seated in a recess 40 with its upper surface 44 facing the lower surface 24 of the flange 26 and with the central perforation 46 coaxial with the aperture 34. The thickness of each spacer 42 can vary depending on how far the lower surface 48 of the spacer 42 is required to protrude below the lower surface 24 of a flange 26. The required amount of protrusion is determined by the dimensions of the gap to be filled between a bone surface and the lower surface 24. A user can, for example, select a spacer 42 of a thickness appropriate to a fill a particular gap. A fastener such as a pedicle screw 36 is disposed through both the aperture 34 of fixation plate 12 and the central perforation 46 of the spacer 42.

The spacers 42 can be made from any compressible material known in the art, which is sufficiently strong to withstand the stresses between fixation plate 12 and a bone surface, and sufficiently durable to last the life of the fixation plate 12 as installed. Elastomeric materials such as polyethylenes, urethanes, and thermoset elastomers such as silicone, are preferred.

The recesses 40 and spacers 42 need not be circular. They can for example be polygonal, as long as the shape of each recess 40 is matched to conform to the shape of its corresponding spacer 42. The recesses 40 need not be situated coaxially with the apertures 34 but can alternatively be eccentric to the apertures 34.

In an alternative embodiment, the recesses 40 are not associated with the apertures 42 and pedicle screws 36 but are situated at any other site on the lower surface 24 that contacts bone surfaces (not shown). In this embodiment, the spacers 42 need not be perforated but can be essentially solid in form.

The spacers 42 can also be utilized in combination with embodiments of the present invention that do not include recesses 40. In this case, the spacers 42 are used the manner of washers, with each spacer 42 disposed flush against the lower surface 24 of a flange 26, each spacer defining a central perforation 46, and each spacer disposed with its perforation 46 coaxial with an aperture 34 (not shown). A fastener such as a pedicle screw 36 is disposed through both the aperture 34 of fixation plate 12 and the central perforation 46 of the spacer 42.

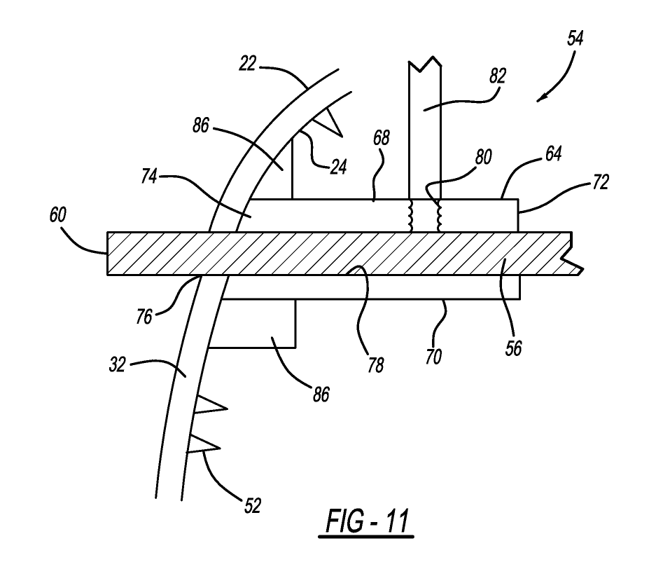

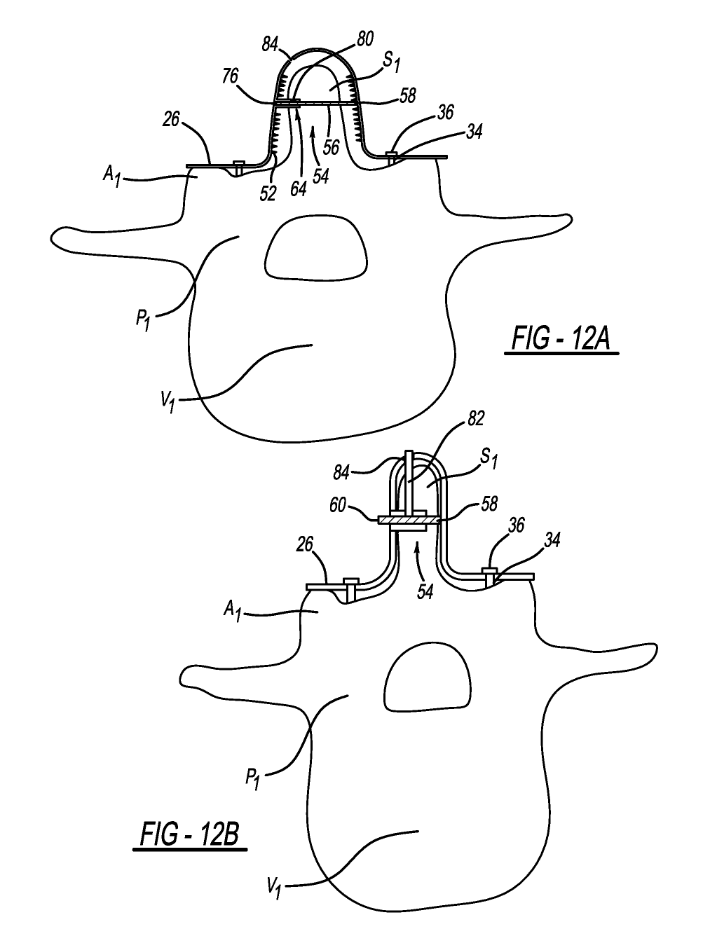

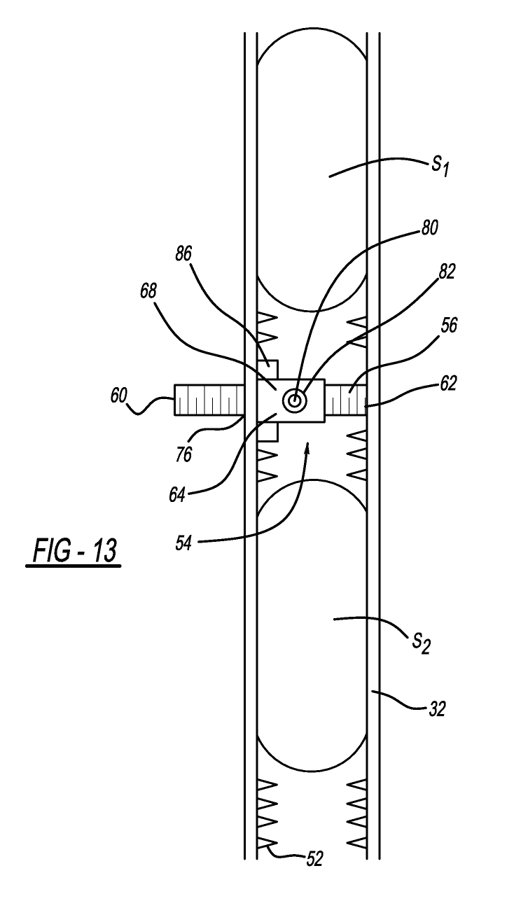

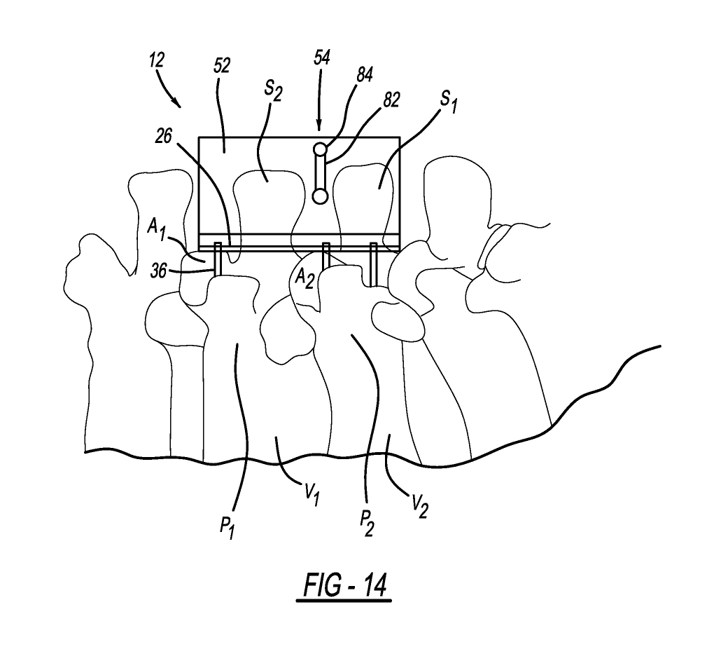

An alternative embodiment of the present invention includes seizing means situated on the lower surface 24 of the fixation plate 12 to seize the surfaces of spinous processes encompassed by the fixation plate 12. In the preferred embodiment, the seizing means includes at least one projection 52, and preferably a plurality of projections 52, extending from the lower surface 24 of the fixation plate 12. In the embodiment illustrated in FIGS. 2B, 10, 11, and 12-14, the projections 52 are spike-like, but they can alternatively take the form of needles, sharpened hooks, multi-pronged burrs, or any other form capable of interacting seizingly with a bone surface. Alternatively, seizing means can be include an adhesive such as a bone cement, for example VERTAPLEX® (Stryker Medical, Portage, Mich.), or double sided adhesive tape.

The projections 52 augment the stability of fixation when spinous processes or remnants thereof are available. In operation, the projections 52 penetrate partially into the surfaces of spinal processes encompassed within the fixation plate 12 to augment the stability and rigidity of the combination of fixation plate 12 with the vertebrae to which it is affixed. The projections 52 and spinal plate can be fabricated as a unit or, alternatively, the projections 52 can be attached separately after fabrication. The projections 52 can be distributed in any density or pattern warranted by the intended site of use and condition of the spinous processes. Preferably, the projections 52 are excluded from the flanges 26 and the apex 30 of the fixation plate 12.

In an alternative embodiment, the fixation plate 12 further includes a gripping subassembly 54 to tighten the shoulders 32 of the fixation plate 12 against spinous processes, or remnants thereof, that are encompassed by the spinal plate 12. Preferably, the previously described projections 52 are also included in this embodiment. The gripping subassembly 54 further augments the rigidity of the fixation of vertebrae by the spinal plate 12.

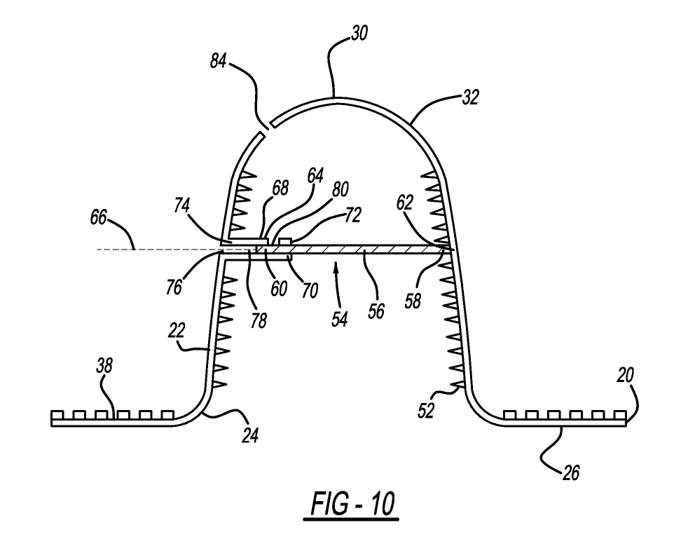

The gripping subassembly 54, illustrated in FIGS. 10-14 includes a bar 56 having an attached end 58 and a free end 60. The attached end 58 of the bar 56 is attached at a bar attachment point 62 situated on the lower surface 24 of the fixation plate 12. Preferably the bar attachment point 62 is situated on a shoulder 32 of the arcuate process 28, approximately midway between a flange 25 and the apex 30 of the fixation plate 12. The attached end 58 of the bar 56 is attached at the bar attachment point 62 by attachment means (not shown) including rivets, a screw threaded socket, or any other means known in the art to yield a physically stable attachment which is not irritating to surrounding tissue.

The free end 60 of the bar 56 is insertable into a bar receiver 64 situated at a point on the lower surface of the fixation plate opposite the attachment point 62. The bar receiver 64, preferably cylindrical, but alternatively rectangular, includes a bar axis 66 parallel to the lateral axis 18 of the spinal plate 12. The bar receiver 64 further includes an upper receiver surface 68 facing the apex 30 of the fixation plate 12, an opposite lower receiver surface 70, an internal end 72 facing the attachment point 62, and an external end 74 defining a bar aperture 76 extending through the upper surface 22 and lower surface 24 of the arcuate process 30. A central bore 78, continuous with the bar aperture 76, extends along the entire extent of the bar axis 66, to receive the bar 56. The upper receiver surface 68 defines a threaded screw hole 80 to receive the distal end of a set screw 82 or other means to clamp the bar 56 in place within the bore 78.

The bar receiver 64 and fixation plate 12 can be fabricated as a single unit, or alternatively the bar receiver can be fabricated separately and affixed to the lower surface 24 of the fixation plate by any affixation means known in the art. The bar receiver 64 can optionally include a broadened base 86 to enhance its stability under the pulling and twisting forces exerted by spinal movements.

The gripping subassembly 54 further includes a set-screw aperture 84 defined in a shoulder of the fixation plate, to permit the insertion of the set screw 82 into the screw hole 80 of the bar receiver 64. The set-screw aperture 84 is situated above, and is coaxial with, the screw hole 80. In use, the bar 56 extends through a space between adjacent spinous processes. This use will often, but not necessarily, require the screw head aperture 84, bar receiver 64, and bar attachment point 62 to be situated asymmetrically with respect to the lateral axis 18 of the fixation plate 12, as illustrated in FIGS. 2B, 4B, and 14.

In operation, the gripping subassembly 54 is preferably tightened about one or more spinous processes after the fixation plate 12 has been anchored into one or more vertebrae as described above. Prior to placement upon the vertebrae, the gripping assembly 54 is in slack condition, which is characterized by the free end 60 of bar 56 being situated within the bore 78 of the bar receiver 64, and the set screw 82 being either absent or situated in the set-screw aperture with its distal end in contact with the screw hole 80 of the bar receiver 64, and its head resting in the set-screw aperture 64.

To tighten the gripping subassembly 54, an operator grasps both shoulders 32 with a pliers or other compressive device. The operator then compresses the shoulders 32 until they abut the lateral surfaces of the spinous processes. If projections 52 are included, they partially penetrate the spinous processes. The compression of the shoulders 32 causes the bar 56 to slide through the bore 78 of bar receiver 64 and can cause the free end 60 of the bar 56 to protrude from the bar aperture 76. At this point the set screw 82 is rotated drive it into the screw hole 80 to exert pressure on the bar 56, locking the bar 56 into place. The gripping subassembly 54 is now in tightened condition.

If it is desirable to eliminate the portion of the free end 60 of the bar 56 that protrudes from the bar aperture 76, then the bar can be provided with periodic scores (not shown) to enable the protruding portion to be readily broken off with a pliers or similar instrument. Alternatively, the bar 56 can be constructed of small adjoining sections interconnected by means of screw threaded sockets (not shown), and the protruding sections can be unscrewed and removed after the installation of fixation plate 12.

The spinal fixation plate 12 of the present invention can be used for spinal stabilization following interbody and/or posterior lumbar fusion. The two primary uses are (1) to provide posterior stability, and (2) to allow fusion by means of grooves on the surface of the device. Suitable uses for the spinal fixation plate include, but are not limited to, as the stabilizing implant used in spinal fusion; (2) to provide motion preservation, specifically of lateral articulation; and (3) for use in conjunction with pedicle screws and rods placed on either side of the fixation plate 12. An example of use of the spinal fixation plate 12 in affixed to vertebrae V1, V2, and V3, conjunction with bone graft material BG, polyaxial pedicle screws, and rods 50 is shown in FIG. 9.

It will be understood that it is within the scope of the present invention to combine features of every alternative embodiment in every possible permutation. For example, a spinal plate 12 including more than four apertures 34, grooves 38, and gripping subassembly 54 is within the scope of the present invention. A spinal plate 12 including a gripping subassembly 54 but not including at least one spike 52 is within the scope of the present invention.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A spinal fixation plate for posterior fixation, comprising a plate body including flattened flanges extending along sides of said plate body, said flanges being interconnected by a convex arcuate process.

2. The spinal fixation plate of claim 1, wherein said convex arcuate process includes an apex at a midpoint of a lateral axis of said plate body.

3. The spinal fixation plate of claim 2, wherein said flanges are flat and oriented parallel to said lateral axis.

4. The spinal fixation plate of claim 3, wherein said convex arcuate process is symmetrical about a longitudinal axis of said plate body.

5. The spinal fixation plate of claim 1, further including at least one aperture for receiving fastening means extending through an upper surface and lower surface of said plate body.

6. The spinal fixation plate of claim 5, wherein said aperture is chosen from the group consisting of a circular hole, oblong hole, slot, elongated slot, through hole, and void.

7. The spinal fixation plate of claim 5, further including grooves on said upper surface.

8. The spinal fixation plate of claim 7, wherein said grooves are seeded with material chosen from the group consisting of bone graft materials, bone growth factors, graft extenders, bulking agents, and combinations thereof.

9. The spinal fixation plate of claim 5, wherein said lower surface further includes seizing means for seizing the surfaces of spinous processes.

10. The spinal fixation plate of claim 1, further including an elastomeric coating.

11. The spinal fixation plate of claim 1, wherein said plate body is arcuately shaped to conform said spinal fixation plate to the curvature of vertebrae.

12. The spinal fixation plate of claim 1, further including cut-outs in said plate body.

13. The spinal fixation plate of claim 1, wherein a lower surface of said flanges includes at least one recess for the seating of at least one compressible spacer.

14. The spinal fixation plate of claim 1, further including gripping means for tightening shoulders of said plate body against spinous processes.

15. A method of fixing vertebrae, including the steps of:
disposing a spinal fixation plate upon dorsal surfaces of adjoining vertebrae, wherein flanges of the spinal fixation plate extend parallel to the spinal column and rest upon superior articular processes of the adjoining vertebrae; and
anchoring the spinal fixation plate to the vertebrae with at least one fastener by disposing the fastener through at least one aperture of the spinal fixation plate into the vertebrae.

16. The method of claim 15, wherein the apertures are situated directly upon the superior articular processes and said disposing step is further defined as disposing the fastener through the dorsal aspect of the superior articular processes and into pedicles.

17. The method of claim 15, prior to said disposing step, further including the step of seeding grooves of the spinal fixation plate with material chosen from the group consisting of bone graft materials, bone growth factors, graft extenders, bulking agents, and combinations thereof, and after said anchoring step, further including the step of inducing bone fusion.

18. The method of claim 17, further including the step of promoting vascularization.

19. The method of claim 15, further including the step of seizing the surfaces of spinous projections and stabilizing the spinal fixation plate.

20. The method of claim 15, further including the step of gripping the spinal fixation plate against surfaces of spinous processes and augmenting the rigidity of fixation of the vertebrae.
